(19) United States
(12) Patent Application Publication
(10) Pub. No.: US 2021/0393828 A1
(43) Pub. Date: Dec. 23, 2021

(54) DISINFECTANT DISPENSER APPARATUS AND METHOD OF USING THE SAME

(71) Applicant: Sandra Garside, Washington, DC (US)

(72) Inventor: Sandra Garside, Washington, DC (US)

(21) Appl. No.: 17/353,350

(22) Filed: Jun. 21, 2021

Related U.S. Application Data
(60) Provisional application No. 63/041,464, filed on Jun. 19, 2020.

(51) Int. Cl. A61L 2/18

(52) U.S. Cl. CPC A61L 2/18; A61L 2202/15; A61L 2202/23

(57) ABSTRACT

A disinfectant dispenser apparatus configured to couple to a beverage container, the apparatus includes a housing having a first end including a through hole, an opposing second end, and a cavity therebetween configured to store a fluid, a pump having plurality of ports including a first port in fluid communication with the cavity, and a button moveably coupled to the pump, wherein when the button is configured to dispense the fluid from the cavity via the through hole when moved from a first position to a second position.

### BACKGROUND OF THE INVENTION

### 1. Field of the Invention

The present general inventive concept relates to a dispenser, and more particularly, to a disinfectant dispenser integrated with a beverage container.

### 2. Background

Many containers exist for containing beverages for drinking. Such containers, such as bottles or cups, have lids with different types of mechanisms for allowing release of the beverage. Examples include a coffee cup with a lid having a sliding valve, and a sports bottle with a lid that screws onto and off from the bottle.

Such bottles and cups are well-known. However, such bottles and cups do not include a section that allows a user to disinfect their hands. For instance, a runner may carry a bottle of water while running. Such a bottle, however, does not allow the runner to disinfect their hands while running outside. This becomes an issue if the runner's hands become dirty, and no bathroom or station is nearby to wash and disinfect their hands.

Other assemblies exist that contain reservoirs for cleaning. However, no such assemblies combine a container of a beverage with a reservoir for cleaning in such a way that is compact and portable.

Therefore, what is needed is a device, or an integrated bottle, that includes both a beverage container for drinking fluids and a disinfectant dispenser for sanitizing a user.

### SUMMARY OF THE INVENTION

Certain of the foregoing and related aspects are readily attained according to the present general inventive concept by providing a disinfectant dispenser apparatus configured to couple to a beverage container, the apparatus includes a housing having a first end including a through hole, an opposing second end, and a cavity therebetween configured to store a fluid, a pump having plurality of ports including a first port in fluid communication with the cavity, and a button moveably coupled to the pump, wherein when the button is configured to dispense the fluid from the cavity via the through hole when moved from a first position to a second position.

The disinfectant dispenser apparatus may further include a coupling member detachably coupled to the housing.

The coupling member may include a first end configured to couple to the housing and an opposing second end configured to receive a conventional beverage container.

An inner surface of the coupling member may include a friction coating to increase friction between the coupling member and the conventional beverage container.

The plurality of ports may include a second port in fluid communication with the through hole of the housing.

The pump may include an integrated one-way valve disposed between the first port and the button.

The one-way valve may prevent the fluid from flowing from the pump through the first port.

Certain of the foregoing and related aspects are readily attained according to the present general inventive concept by also providing an integrated disinfectant dispenser apparatus including a housing having a first end including a through hole, an opposing second end, and a cavity therebetween configured to store a fluid, a pump having plurality of ports including a first port in fluid communication with the cavity, and a button moveably coupled to the pump, wherein when the button is configured to dispense the fluid from the cavity via the through hole when moved from a first position to a second position.

Certain of the foregoing and related aspects are readily attained according to the present general inventive concept by also providing a vessel, for containing a beverage and for dispensing a disinfectant, including an outer body having an outer wall, an inner wall, an interior space, a bottom closed end, and a top open end having a plurality of threads, an inner body having an outer wall, an inner wall, an interior space, a bottom closed end, and a top open end, wherein the inner body is located within the interior space of the outer body, a cap having a bottom section, having a plurality of corresponding threads for mating with the plurality of threads of the top open end of the outer body, and a top section having an interior space including an assembly for dispensing the disinfectant, the assembly including: a valve body having a first outlet opposite a second outlet, and a third outlet at an angle to the first outlet and the second outlet, a first check ball between the first outlet of the valve body and a valve nozzle, a second check ball between the second outlet of the valve body and a valve cap, a dispenser, for releasing the disinfectant from the vessel, positioned around the third outlet of the valve body, a reservoir, for containing the disinfectant, positioned above the valve cap and having a rim including a plurality of threads, and a lid having a rim including a plurality of corresponding threads for mating with the plurality of threads of the reservoir.

The third outlet of the valve body is positioned normal to both the first outlet and the second outlet of the valve body.

The interior space of the inner body contains the beverage. The cap is removed from the outer body to allow for drinking of the beverage.

A gap is formed between the inner wall of the outer body and the outer wall of the inner body for providing insulation to the beverage.

In contrast with conventional methods disclosed by the prior art, the present general inventive concept utilizes a comparison of parameters from an intra-personal point-of-view and parameters from an inter-personal point-of-view to assist an individual to improve their self-esteem, confidence, and public speaking skills. Specifically, one focuses more on issues that one controls, and less on issues that others control.

### Definitions

When a first component is defined as being “integrated” or “integral” with a second component, it is meant that the two components are connected together and cannot be separated without damaging or destroying one or both components and/or their functions. The components described as being integrated are connected substantially permanently mechanically or via an adhesive or by any other such means.

### BRIEF DESCRIPTION OF THE DRAWINGS

These and/or other aspects of the present general inventive concept will become apparent and more readily appreciated from the following description of the embodiments, taken in conjunction with the accompanying drawings of which:

FIG. 1 illustrates a perspective view of a disinfectant dispenser apparatus according to an example embodiment of the present general inventive concept;

FIG. 2 illustrates a front view of the disinfectant dispenser apparatus illustrated FIG. 1;

FIG. 3 illustrates a side view of the disinfectant dispenser apparatus illustrated in FIG. 1;

FIG. 4 illustrates a top view of the disinfectant dispenser apparatus illustrated in FIG. 1;

FIG. 5A illustrates a partial exploded assembly perspective view of the disinfectant dispenser apparatus illustrated in FIG. 1;

FIG. 5B illustrates a complete exploded assembly perspective view of the disinfectant dispenser apparatus illustrated in FIG. 1, showing hidden lines;

FIG. 5C illustrates a side view of the disinfectant dispenser apparatus illustrated in FIG. 5B;

FIG. 6A illustrates a side view of the pump illustrated in FIG. 5C with a button disposed at a first position;

FIG. 6B illustrates a side view of the pump illustrated in FIG. 5C with a button disposed at a middle position;

FIG. 6C illustrates a side view of the pump illustrated in FIG. 5C with a button disposed at a second position;

FIG. 7 illustrates a side view of the disinfectant dispenser apparatus illustrated in FIG. 3, showing hidden lines;

FIG. 8 illustrates a side view of the disinfectant dispenser apparatus illustrated in FIG. 7 and a conventional beverage container;

FIG. 9 illustrates a side view of the disinfectant dispenser apparatus illustrated in FIG. 7 assembled with a conventional beverage container;

FIG. 10 illustrates a perspective view of an integrated disinfectant dispenser apparatus, according to another example embodiment of the present general inventive concept;

FIG. 11 illustrates a side view of the integrated disinfectant dispenser apparatus illustrated in FIG. 10;

FIG. 12 illustrates a top view of the integrated disinfectant dispenser apparatus illustrated in FIG. 10;

FIG. 13 illustrates an exploded assembly view of the integrated disinfectant dispenser apparatus illustrated in FIG. 10; and

FIG. 14 illustrates a cross-section of the integrated disinfectant dispenser apparatus illustrated in FIG. 12, taken along line A-A.

### DESCRIPTION OF THE PREFERRED EMBODIMENTS

The present inventive concept will now be described more fully with reference to the accompanying drawings, in which exemplary embodiments of the present general inventive concept are illustrated. The inventive concept may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals refer to like elements throughout.

The present general inventive concept includes a disinfectant dispenser configured to be detachably coupled to a conventional beverage container.

The present general inventive concept also includes a beverage container integrally formed with a disinfectant dispenser. The integrated beverage container and disinfectant dispenser according to the present invention is designed as a convenient, dual purpose tool that provides both an insulated (or a non-insulated) drinking container with an integrated disinfectant dispenser designed to dispense a disinfectant fluid. However, the present general inventive concept is not limited thereto.

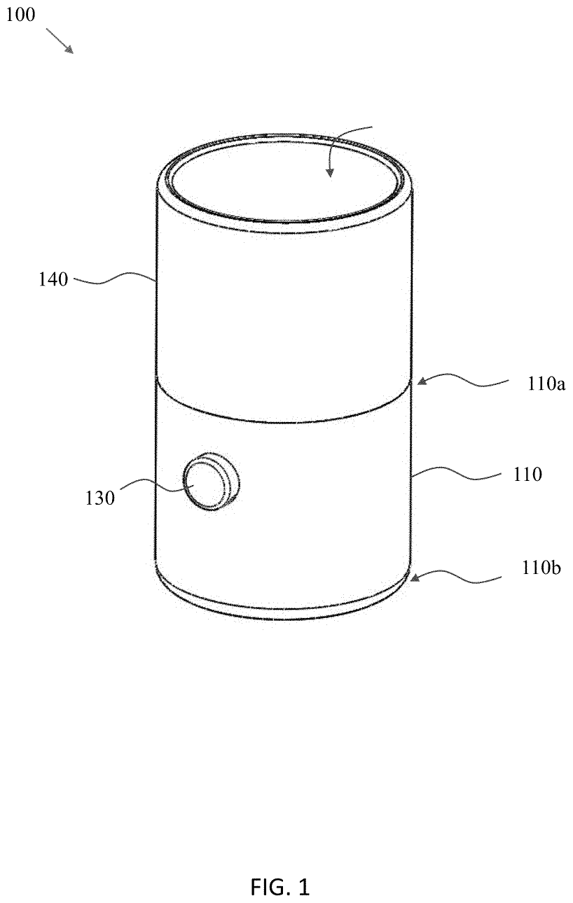

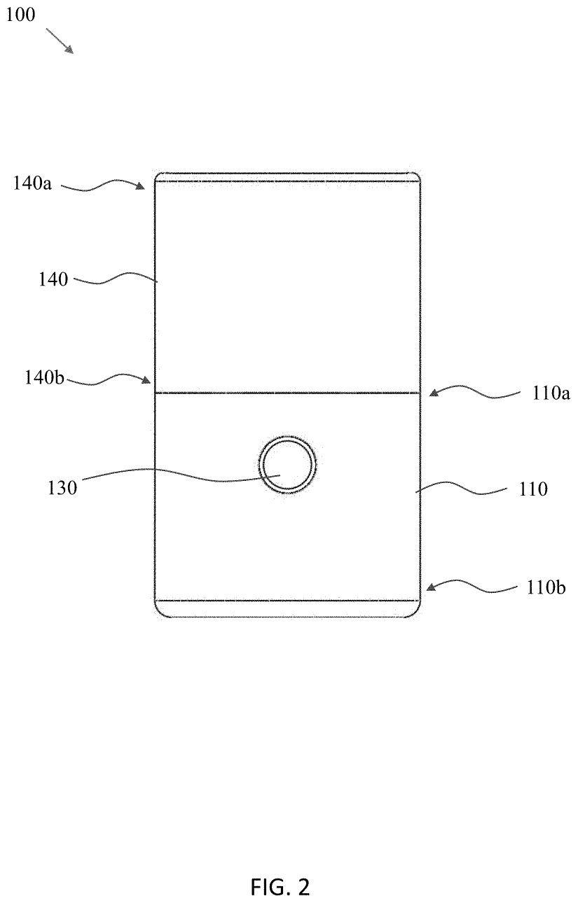

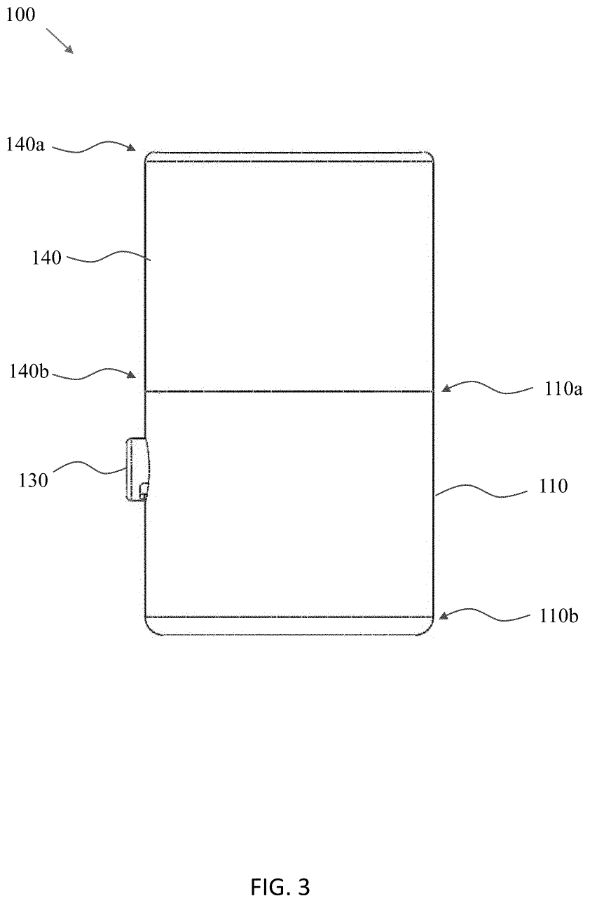

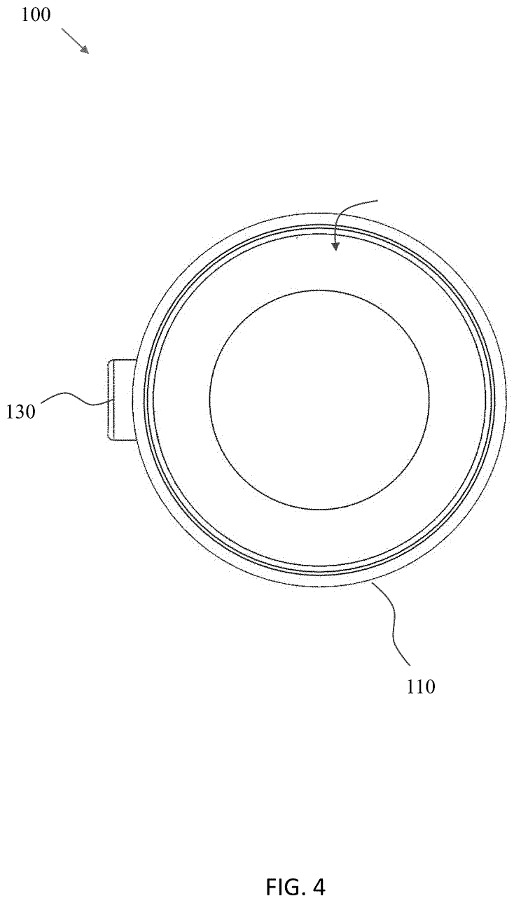

FIG. 1 illustrates a perspective view of a disinfectant dispenser apparatus 100 according to an example embodiment of the present general inventive concept, FIG. 2 illustrates a front view of the disinfectant dispenser apparatus 100 illustrated FIG. 1, and FIG. 3 illustrates a side view of the disinfectant dispenser apparatus 100 illustrated in FIG. 1. FIG. 4 illustrates a top view of the disinfectant dispenser apparatus 100 illustrated in FIG. 1.

Referring to FIGS. 1 through 4, according to an exemplary embodiment of the present general inventive concept, the disinfectant dispenser apparatus 100 is configured to be detachably coupled to a conventional beverage container 10.

In the present exemplary embodiment, the disinfectant dispenser apparatus 100 includes a housing member 110 having a cavity 112 formed therein that is designed to store a fluid such as a disinfectant, a pump 120 that is configured to dispense the fluid stored within the cavity 112, and a button 130 that is movably coupled to the pump 120 and configured to activate the pump 120 to dispense the fluid when moved from a first position P1 to a different second position P2. However, the present general inventive concept is not limited thereto.

In the present embodiment, the housing member 110 includes a first end 110a and an opposing second end 110b. The cavity 112 is configured to store a fluid, such as a disinfectant. However, the present general inventive concept is not limited thereto. That is, in alterative embodiments, the cavity 112 may be configured to store and dispense disinfectant wipes.

In the present embodiment, the disinfectant dispenser apparatus 100 includes a coupling member 140 that may be detachably coupled to the housing member 110. The coupling member 140 includes a first end 140a configured to receive a conventional beverage container 10 and an opposing second end 140b configured to couple to the housing member 110. However, the present general inventive concept is not limited thereto.

In the present embodiment, the coupling member 140 is detachably coupled to the housing member 110 and includes a first end 140a configured to couple to the housing member 110 and an opposing second end 140b configured to receive a conventional beverage container 10.

In alternative embodiments, an inner surface of the coupling member 140 includes a friction coating to increase friction between the coupling member 110 and the conventional beverage container 10.

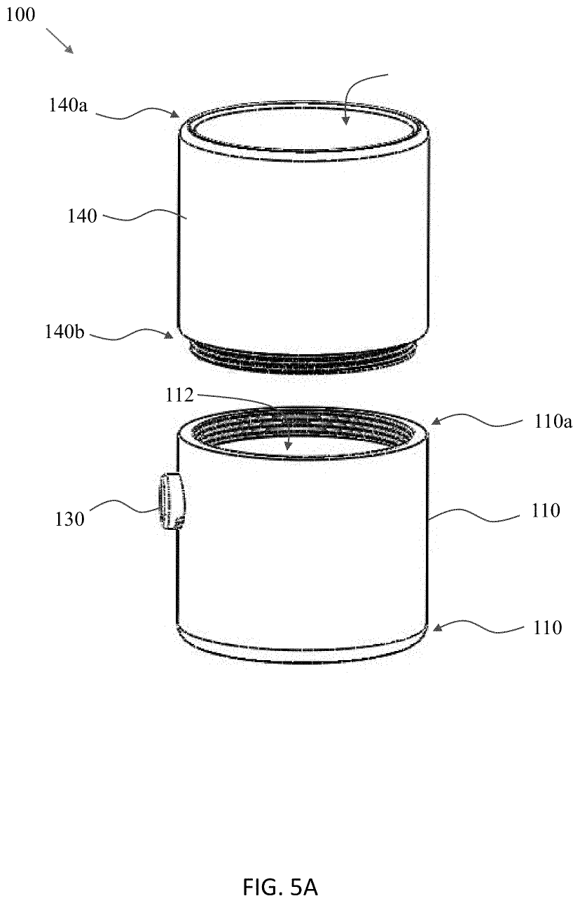

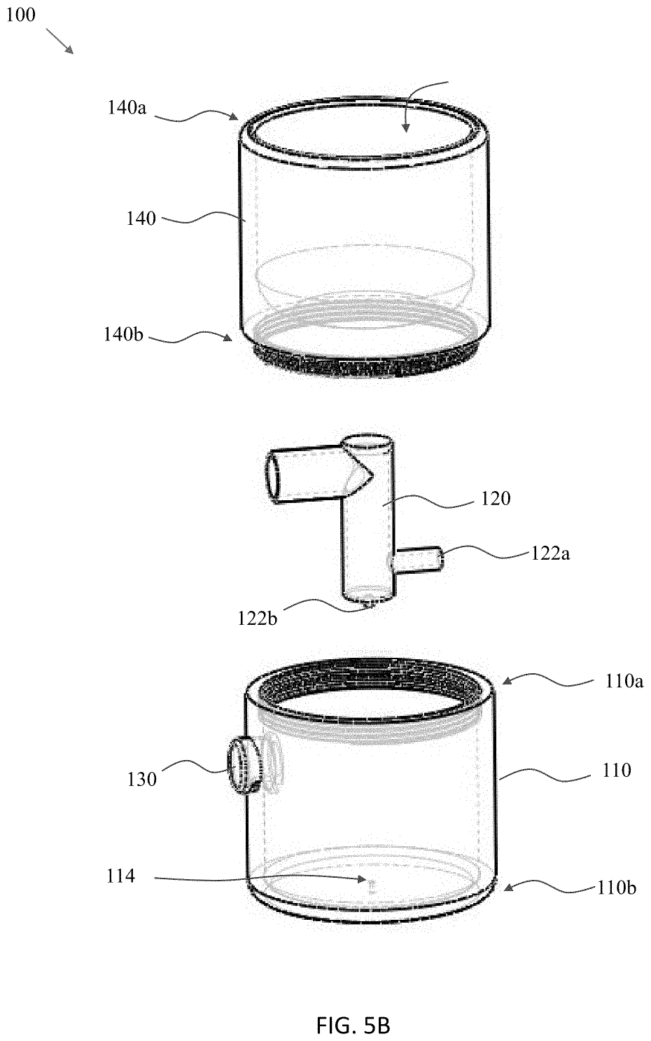

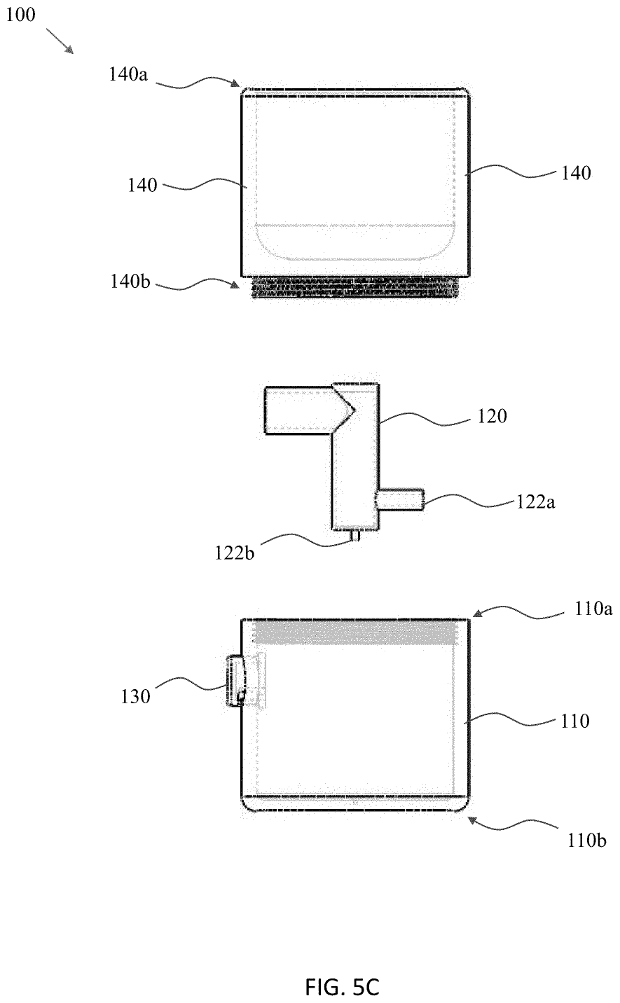

Referring to FIG. 5A, in the present embodiment, the second end 140b of the coupling member 140 includes a plurality of threads 141 configured to detachably couple to the first end of the housing member 110.

Referring to FIG. 5B, in the present embodiment, the plurality of ports 122 of the pump 120 include a first port 122a in fluid communication with the cavity 112 and a second port 122b configured to dispense the fluid through a through hole 114 in the housing member 110.

In the present embodiment, the plurality of ports 122 of the pump 120 includes a first port 122a (i.e., inlet) designed and/or configured to be in fluid communication with the cavity 112 and a second port 122b (i.e., outlet) designed and/or configured to be in fluid communication with the second end 110b of the housing member 110.

In the present embodiment, the button 130 is designed and/or configured to move from a first position P1 to a middle position M, and then to a second position P2 in order to generate a vacuum within the pump 120 to draw the fluid stored within the cavity 112 into the pump 120 through the first port 122a and then to dispense the fluid out through the second port 122b.

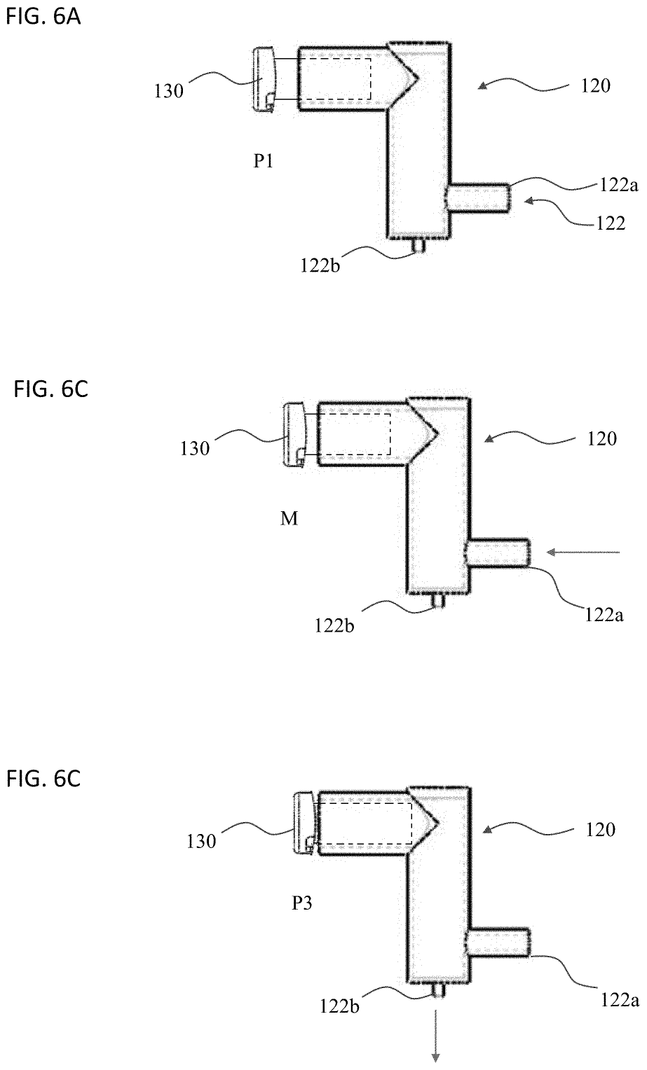

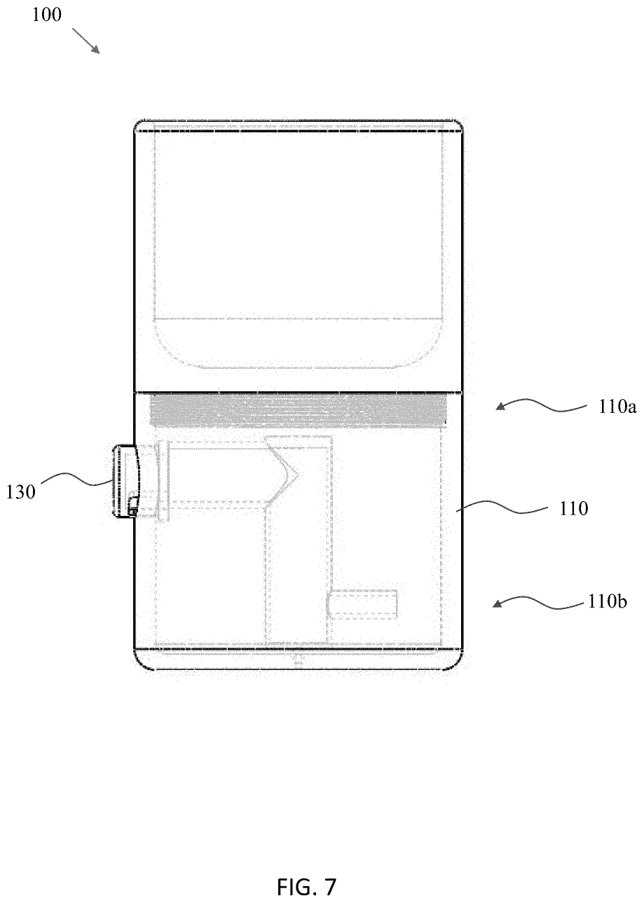

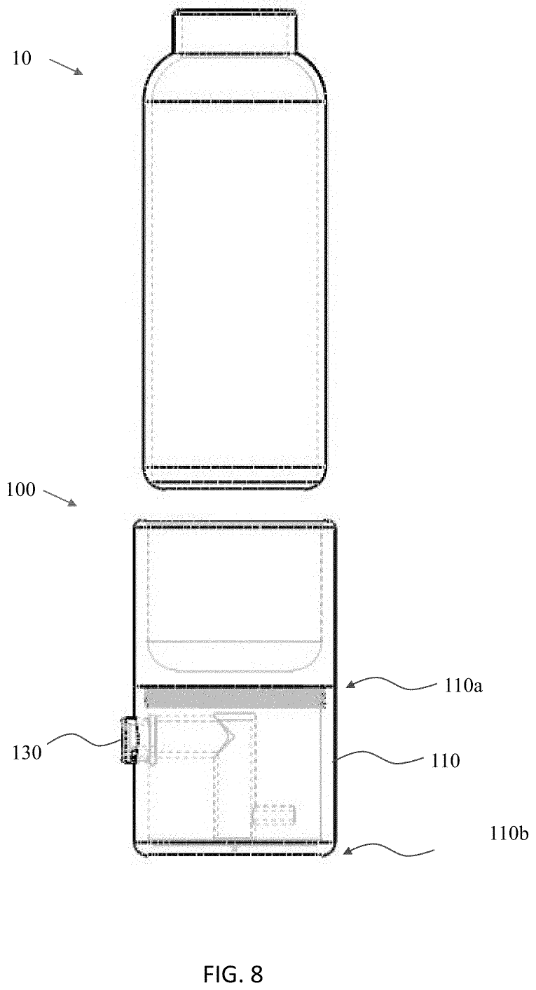

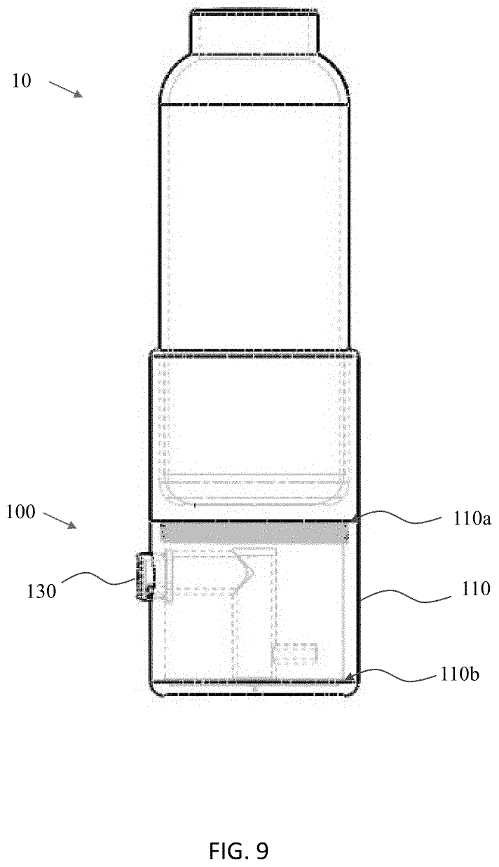

Referring to FIG. 6A, when the button 130 is disposed at the first position P1, the pump 120 is empty of fluid.

Referring to FIG. 6B, when the button 130 is moved from the first position P1 to the middle position M, a vacuum is created within the pump 120 to draw in fluid from the cavity 112 through the first port 122a.

Referring to FIG. 6C, when the button 130 is moved from the middle position M, the fluid drawn into the pump 120 is dispensed out of the pump 120 through the second port 122b and the through hole 114 of the housing member 110.

In alternative embodiments, the pump includes an integrated one-way valve disposed between the first port and the button to prevent fluid from escaping the pump through the first port 122a. However, the present general inventive concept is not limited thereto.

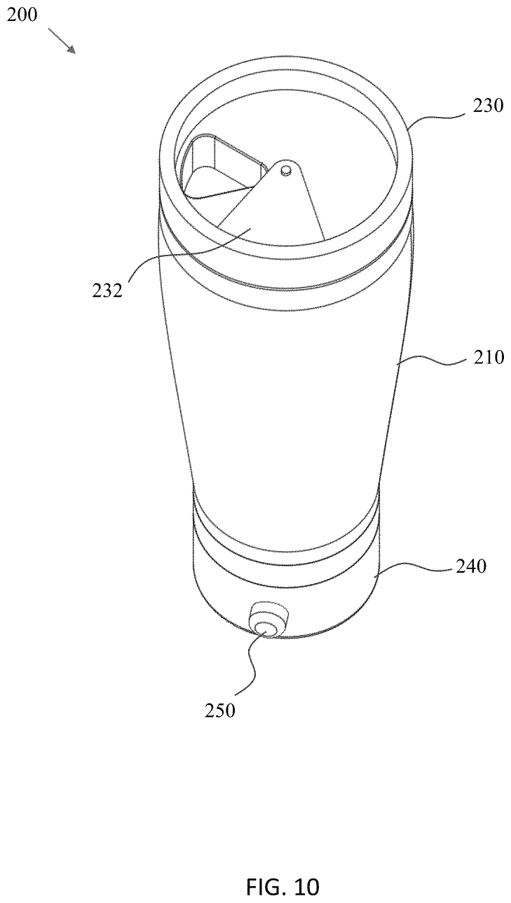

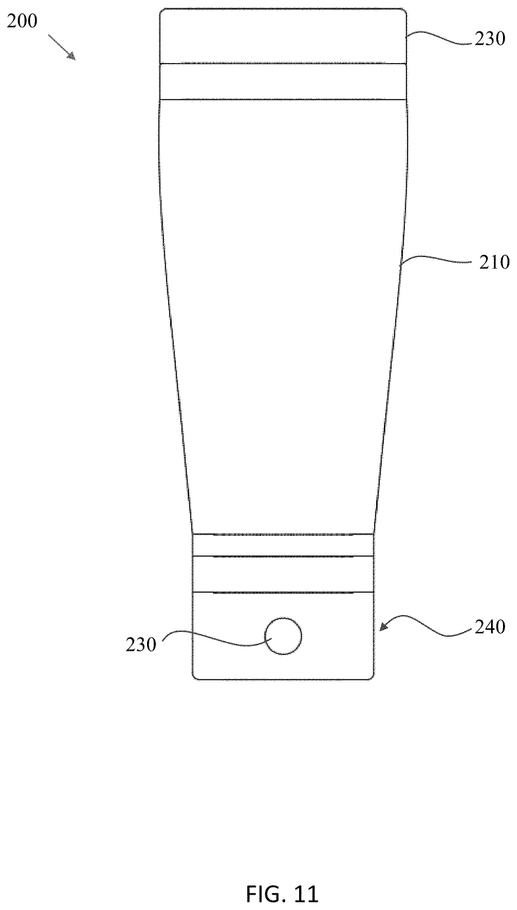

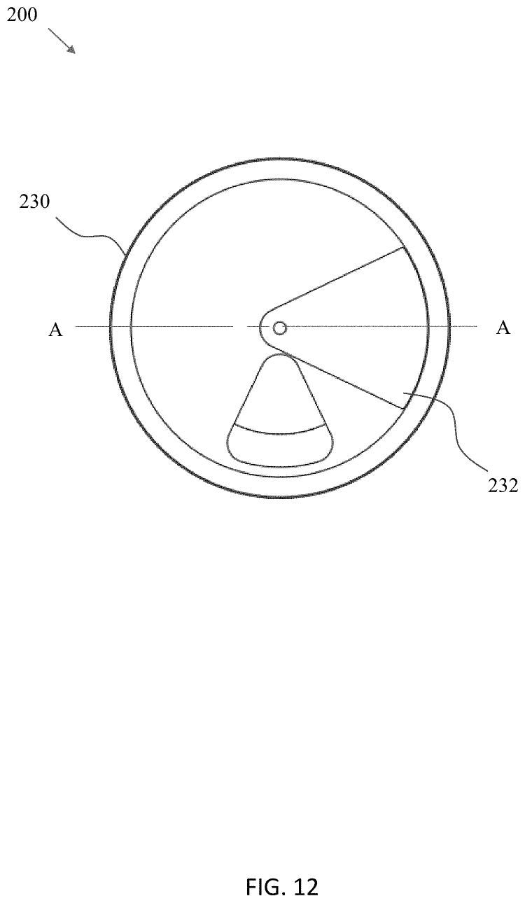

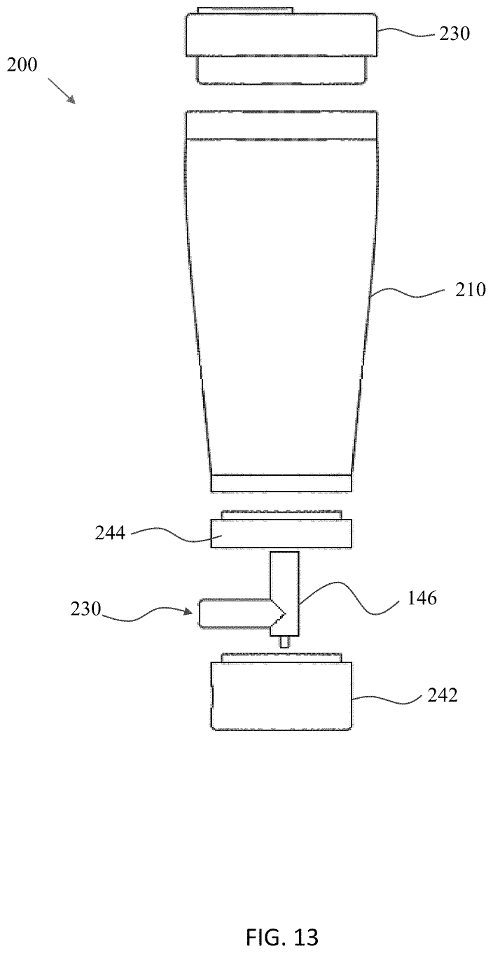

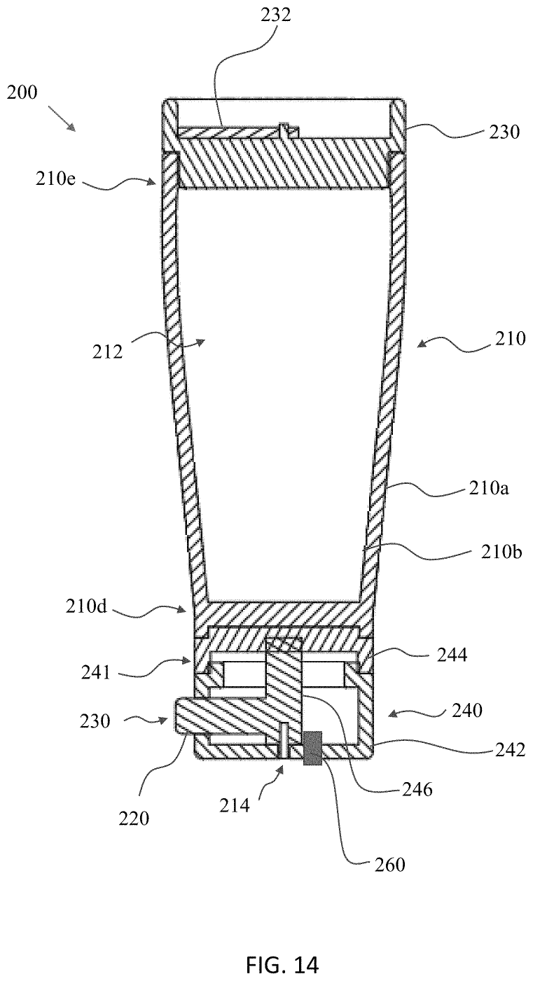

FIG. 10 illustrates a perspective view of an integrated disinfectant dispenser apparatus 200, according to another example embodiment of the present general inventive concept, FIG. 11 illustrates a side view of the integrated disinfectant dispenser apparatus 200 illustrated in FIG. 10, and FIG. 12 illustrates a top view of the integrated disinfectant dispenser apparatus 200 illustrated in FIG. 10. FIG. 13 illustrates an exploded assembly view of the integrated disinfectant dispenser apparatus 200 illustrated in FIG. 10 and FIG. 14 illustrates a cross-section of the integrated disinfectant dispenser apparatus 200 illustrated in FIG. 12, taken along line A-A.

Referring to FIG. 10, in the present embodiment of the present general inventive concept, the integrated drinking vessel and disinfectant dispenser 200 includes an outer body 210, an inner beverage container 220, a removable lid 230 with a movable valve 232, and an integrated disinfectant dispenser 240.

As depicted in FIGS. 10 to 14, the outer body 210 has an outer wall 210a, an inner wall 210b, an interior space 210c, a bottom closed end 210d, and a top open end 210e.

The present embodiment is substantially similar to the previous embodiment except for providing a drinking vessel/container integrally formed with a disinfectant dispenser configured to dispense a fluid, such as a disinfectant. However, the present general inventive concept is not limited thereto. That is, in alternative embodiments, the disinfectant dispenser 240 may be configured to dispense a disinfectant wipe and/or emit a disinfectant light 260, such as an ultraviolet light (see FIG. 14).

In the present exemplary embodiment, the integrated disinfectant dispenser apparatus 200 includes a housing member 210 having a cavity 212 formed therein that is designed to store a fluid such as a disinfectant, a pump 220 having a plurality of ports 222 configured to dispense the stored fluid, and a button 230 movably coupled to the pump 220 and configured to activate the pump 220 to dispense the fluid stored in the cavity 212. However, the present general inventive concept is not limited thereto.

In alternative embodiments, the bottom end 210d includes a coupling member 241 configured to detachably couple the housing member 210 to the disinfectant dispenser 240. That is, the coupling member 244 includes a first end 244a configured to couple to the housing member 210 and an opposing second end 244b configured to couple to the disinfectant dispenser 240.

According to the present invention, the combined drinking vessel and integrated disinfectant dispenser also includes an inner body. The inner body has an outer wall, an inner wall, an interior space, a bottom closed end, and a top open end. The inner body is located within the interior space of the outer body to thereby form an insulated beverage container. In an example embodiment, the interior space of the inner body contains the beverage, such as coffee or water.

In alternative embodiments, a gap may be formed between the inner wall of the outer body and the outer wall of the inner body for providing insulation to the beverage.

In an example embodiment, both the outer body and the inner body are made of polymer materials. Non-limiting examples of such polymer materials include BPA-free polypropylene, polyethylene, and polycarbonate. The outer body and the inner body can be formed, for instance, by injection molding. However, the present general inventive concept is not limited thereto.

In an example embodiment, the combined drinking vessel and integrated disinfectant dispenser 100 includes a removable lid 230 with a movable valve that is detachably coupled to the outer housing member. The valve nay be moved between a first position to seal off the interior space of the inner beverage container and a second position to open the interior space of the beverage container 120.

In the present embodiment, the combined drinking vessel and integrated disinfectant dispenser 200 further includes an integrated disinfectant dispenser 240 coupled to a bottom end of the housing member 210.

The integrated disinfectant dispenser 240 includes a dispenser housing 242 to store a disinfectant, a disinfectant housing cap 244 and a dispenser 146 disposed within the dispenser housing 142. The dispenser 146 includes a push-button 148 which, when pressed, dispenses a disinfectant through the dispensing valve 148a.

In operation, a user may remove the integrated disinfectant dispenser from the bottom end of the outer housing member 110. Next, the user may remove the housing cap 144 from the dispenser housing in order to refill the dispenser housing with a disinfectant or other type of fluid.

In the present embodiment, the integrated disinfectant dispenser 140 includes a first check ball (for instance, a 316 stainless steel ball bearing) between the first outlet of the valve body and a valve nozzle (for instance, a 316 stainless steel check spring) a second check ball between the second outlet of the valve body and a valve cap (for instance, a 316 stainless steel plunge spring).

The present inventive concept includes an integrated dispenser for releasing the disinfectant from the vessel. The dispenser may include a button, knob, or lever that, when turned in a first direction (or pressed), opens the assembly, thereby causing the release of the disinfectant.

Conversely, when the lever is turned in a second direction, the dispenser closes off the assembly, thereby preventing the release of the disinfectant.

The dispenser includes a reservoir, for containing and storing the disinfectant, that is positioned above the valve cap and has a rim including a plurality of threads.

In an example embodiment, the reservoir is lined with copper. Thus, the reservoir is copper-rich to create an environment that disinfects itself. However, the present general inventive concept is not limited thereto.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

What is claimed is:

1. A disinfectant dispenser apparatus configured to couple to a beverage container, the apparatus comprising:
a housing member having a first end including a through hole, an opposing second end, and a cavity therebetween configured to store a fluid;
a pump having plurality of ports including a first port in fluid communication with the cavity; and
a button moveably coupled to the pump,
wherein when the button is configured to dispense the fluid from the cavity via the through hole when moved from a first position to a second position.

1. The disinfectant dispenser apparatus of claim 1, further comprising a coupling member detachably coupled to the housing.

2. The disinfectant dispenser apparatus of claim 2, wherein the coupling member includes a first end configured to couple to the housing and an opposing second end configured to receive a conventional beverage container.

3. The disinfectant dispenser apparatus of claim 3, wherein an inner surface of the coupling member includes a friction coating to increase friction between the coupling member and the conventional beverage container.

4. The disinfectant dispenser apparatus of claim 3, wherein the plurality of ports include a second port in fluid communication with the through hole of the housing.

5. The disinfectant dispenser apparatus of claim 1, wherein the pump includes an integrated one-way valve disposed between the first port and the button.

6. The disinfectant dispenser apparatus of claim 1, wherein the one-way valve prevents the fluid from flowing from the pump through the first port.

7. The disinfectant dispenser apparatus of claim 1, wherein the fluid includes a disinfectant liquid.

8. An integrated disinfectant dispenser apparatus comprising:
a housing member having a first end including a through hole, an opposing second end, and a cavity therebetween configured to store a fluid;
a pump having plurality of ports including a first port in fluid communication with the cavity; and
a button moveably coupled to the pump,
wherein when the button is configured to dispense the fluid from the cavity via the through hole when moved from a first position to a second position.

9. The integrated disinfectant dispenser apparatus of claim 9, further comprising a coupling member detachably coupled to the housing.

10. The disinfectant dispenser apparatus of claim 10, wherein the coupling member includes a first end configured to couple to the housing and an opposing second end configured to receive a conventional beverage container.

11. The disinfectant dispenser apparatus of claim 11, wherein an inner surface of the coupling member includes a friction coating to increase friction between the coupling member and the conventional beverage container.

12. The disinfectant dispenser apparatus of claim 11, wherein the plurality of ports include a second port in fluid communication with the through hole of the housing.

13. The disinfectant dispenser apparatus of claim 9, wherein the pump includes an integrated one-way valve disposed between the first port and the button.

14. The disinfectant dispenser apparatus of claim 9, wherein the one-way valve prevents the fluid from flowing from the pump through the first port.

15. The disinfectant dispenser apparatus of claim 9, wherein the fluid includes a disinfectant liquid.
